(19) United States
(12) Patent Application Publication
(10) Pub. No.: US 2015/0173723 A1
(43) Pub. Date: Jun. 25, 2015

(54) METHOD AND SYSTEM FOR AUTOMATIC NEEDLE RECALIBRATION DETECTION

(71) Applicant: General Electric Company, Schenectady, NY (US)

(72) Inventors: David J. Bates, Wauwatosa, WI (US); Menachem Halmann, Wauwatosa, WI (US)

(73) Assignee: General Electric Company, Schenectady, NY (US)

(21) Appl. No.: 14/136,865

(22) Filed: Dec. 20, 2013

(51) Int. Cl. A61B 8/00; A61B 19/00; A61B 5/06; A61B 8/14; A61B 8/08; A61B 17/34

(52) U.S. Cl. CPC A61B 8/58; A61B 8/0841; A61B 17/3478; A61B 8/467; A61B 8/461; A61B 5/062; A61B 8/14; A61B 19/54; A61B 19/5225; A61B 2019/5458; A61B 2019/5276

(57) ABSTRACT

A tracking system that includes a sensor and emitter is calibrated, and a tracked position and orientation of a surgical instrument is determined based at least in part on tracking information emitted by the emitter and detected by the sensor. An ultrasound system performs an ultrasound scan to acquire ultrasound scan data including the surgical instrument. The ultrasound system determines a scanned position and orientation of the surgical instrument based on the ultrasound scan data. The ultrasound system compares the tracked position and orientation with the scanned position and orientation to determine a calibration error. If the calibration error exceeds a threshold, the ultrasound system may (1) prompt a user to repeat the tracking system calibration step, (2) automatically recalibrate the tracking system and/or the ultrasound system, or (3) provide a user option for proceeding with automatic recalibration of the tracking system and/or the ultrasound system.

### CROSS-REFERENCE TO RELATED APPLICATIONS/INCORPORATION BY REFERENCE

[Not Applicable]

### FIELD OF THE INVENTION

Certain embodiments of the invention relate to ultrasound imaging and surgical instrument tracking. More specifically, certain embodiments of the invention relate to a method and system for automatic needle recalibration detection by comparing a recognized needle position and orientation in ultrasound data with a tracked needle position and orientation provided by a tracking system.

### BACKGROUND OF THE INVENTION

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image.

In conventional ultrasound imaging, an operator of an ultrasound system can acquire images in various modes, such as a non-compounding mode and compounding modes that may include electronically steering left or right (in 2D) or left, right, in, or out (in 3D). The term “compounding” generally refers to non-coherently combining multiple data sets to create a new single data set. The plurality of data sets may each be obtained from imaging the object from different angles, using different imaging properties, such as, for example, aperture and/or frequency, and/or imaging nearby objects (such as slightly out of the plane steering). These compounding techniques may be used independently or in combination to improve image quality.

Ultrasound imaging may be useful in positioning an instrument at a desired location inside a human body. For example, in order to perform a biopsy on a tissue sample, it is important to accurately position a biopsy needle so that the tip of the biopsy needle penetrates the tissue desired to be sampled. By viewing the biopsy needle in real time using an ultrasound imaging system, the biopsy needle can be directed toward the target tissue and inserted to the required depth. Thus, by visualizing both the tissue to be sampled and the penetrating instrument, accurate placement of the instrument relative to the tissue can be achieved.

A conventional biopsy needle is a specular reflector, meaning that it behaves like a mirror with regard to the ultrasound waves reflected off of it. The ultrasound is reflected away from the needle at an angle equal to the angle between the transmitted ultrasound beam and the needle. Ideally, an incident ultrasound beam would be substantially perpendicular with respect to a surgical needle in order to visualize the needle most effectively. The smaller the angle at which the needle is inserted relative to the axis of the transducer array, i.e., the imaginary line normal to the face of the transducer array, the more difficult it becomes to visualize the needle. In a typical biopsy procedure using a linear probe and conventional needle, the geometry is such that most of the transmitted ultrasound energy is reflected by the needle away from the transducer array face and thus is poorly detected by the ultrasound imaging system and may be difficult for the operator to recognize.

In some cases, electronic steering can improve visualization of a surgical needle by increasing an angle at which a transmitted ultrasound beam impinges upon the needle, which increases the system's sensitivity to the needle because the reflection from the needle is directed closer to the transducer array. A composite image of the needle can be made by acquiring a frame using a linear transducer array operated to scan without steering (i.e., with beams directed normal to the array) and one or more frames acquired by causing the linear transducer array to scan with beams steered toward the needle. The component frames are combined into a compound image by summation, averaging, peak detection, or other combinational means. The compounded image may display enhanced specular reflector delineation compared to a non-compounded ultrasound image, which serves to emphasize structural information in the image.

Ultrasound imaging system operators often rely upon technology when performing a medical procedure, such as a biopsy procedure. A tracking system may provide positioning information for the needle with respect to the patient, a reference coordinate system, or the ultrasound probe, for example. An operator may refer to the tracking system to ascertain the position of the needle even when the needle is not within the region or volume of tissue currently being imaged and displayed. As such, the tracking or navigation system allows the operator to visualize the patient's anatomy and better track the position and orientation of the needle. The operator may use the tracking system to determine when the needle is positioned in a desired location such that the operator may locate and operate on a desired or injured area while avoiding other structures. Increased precision in locating medical instruments within a patient may provide for a less invasive medical procedure by facilitating improved control over smaller instruments having less impact on the patient. Improved control and precision with smaller, more refined instruments may also reduce risks associated with more invasive procedures such as open surgery.

Tracking systems may be electromagnetic or optical tracking systems, for example. Electromagnetic tracking systems may employ a permanent magnet as an emitter and a sensor as a receiver, or can employ coils as receivers and transmitters. Magnetic fields generated by the permanent magnet(s) or transmitter coil(s) may be detected by the sensor(s) or receiver coil(s) and used to determine position and orientation information of a surgical instrument, for example. Prior to performing a medical procedure, the tracking system is calibrated. For example, in a tracking system comprising a permanent magnet emitter coupled to or within a surgical needle and one or more sensors coupled to or within a probe, the needle may be removed from the surgical environment so that the tracking system can be calibrated to remove or zero-out ambient magnetic fields detected by the sensor(s). However, a subsequent change of magnetic field in the procedure room (e.g., introduction of a metallic object) or even a slight movement (e.g., a rotation) of the hand-held ultrasound probe during a procedure can cause positioning errors in the tracking system, which may necessitate recalibration of the tracking system. In known tracking systems that use permanent magnets, for example, recalibration is typically performed by removing the surgical instrument that includes the emitter from the surgical environment, which could be inconvenient when the surgical instrument is within a patient, for example.

Further limitations and disadvantages of conventional and traditional approaches will become apparent to one of skill in the art, through comparison of such systems with some aspects of the present invention as set forth in the remainder of the present application with reference to the drawings.

### BRIEF SUMMARY OF THE INVENTION

A system and/or method is provided for automatic needle recalibration detection, substantially as shown in and/or described in connection with at least one of the figures, as set forth more completely in the claims.

These and other advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to provide automatic needle recalibration detection by comparing a recognized needle position and orientation in ultrasound data with a tracked needle position and orientation provided by a tracking system, in accordance with an embodiment of the invention.

FIG. 2 is a flow chart illustrating exemplary steps that may be utilized for providing automatic needle recalibration detection by comparing a recognized needle position and orientation in ultrasound data with a tracked needle position and orientation provided by a tracking system, in accordance with an embodiment of the invention.

### DETAILED DESCRIPTION OF THE INVENTION

Certain embodiments of the invention may be found in a method and system for providing automatic needle recalibration detection by comparing a recognized needle position and orientation in ultrasound data with a tracked needle position and orientation provided by a tracking system.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments of the present invention. The following detailed description is, therefore, not to be taken in a limiting sense, and the scope of the present invention is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “an embodiment,” “one embodiment,” “a representative embodiment,” “an exemplary embodiment,” “various embodiments,” “certain embodiments,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode, CF-mode and/or sub-modes of CF such as TVI, Angio, B-flow, BMI, BMI_Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations needed for the invention, such as single or multi-core: CPU, Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

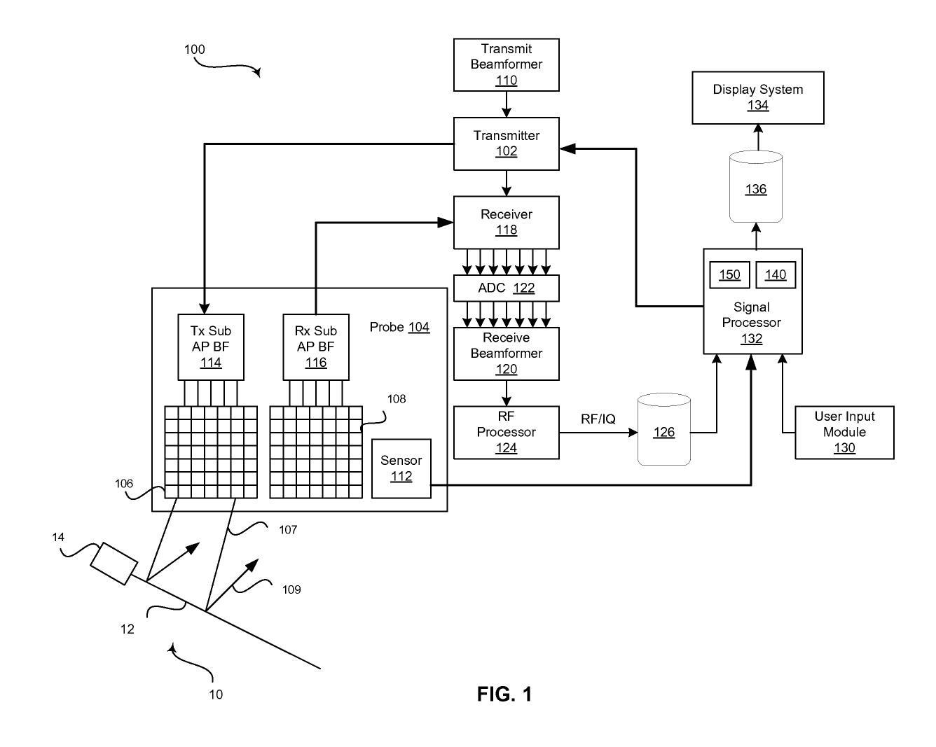

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to provide automatic needle recalibration detection by comparing a recognized needle 10 position and orientation in ultrasound data 109 with a tracked needle 10 position and orientation provided by a tracking system 14, 112, in accordance with an embodiment of the invention. Referring to FIG. 1, there is shown a surgical instrument 10 and an ultrasound system 100. The surgical instrument 10 can be a surgical needle that comprises a needle portion 12 and a needle emitter 14. Notwithstanding, the invention is not limited in this regard. Accordingly, in some embodiments of the invention, the surgical instrument may be any suitable surgical instrument. The ultrasound system 100 comprises a transmitter 102, an ultrasound probe 104, a transmit beamformer 110, a receiver 118, a receive beamformer 120, a RF processor 124, a RF/IQ buffer 126, a user input module 130, a signal processor 132, an image buffer 136, and a display system 134.

The surgical needle 10 comprises a needle portion 12 that includes a distal insertion end and a proximal hub end. A needle emitter 14 is attached to the needle portion 12 at the proximal hub end and/or is secured within a housing attached to the proximal hub end of the needle portion 12. The needle emitter 14 can correspond with a probe sensor 112 of the ultrasound system 100 probe 104, for example. The emitter may be a permanent magnet that corresponds with a sensor, an electromagnetic coil that corresponds with a receiver, an optical source that corresponds with a photo-detector, or any suitable emitter that corresponds with a sensor to form a tracking system. As an example, the needle emitter 14 may comprise a magnetic element that generates a magnetic field detectable by one or more sensors of the probe sensor 112 to enable the position and orientation of the surgical needle 10 to be tracked by the ultrasound system 100.

The transmitter 102 may comprise suitable logic, circuitry, interfaces and/or code that may be operable to drive an ultrasound probe 104. The ultrasound probe 104 may comprise suitable logic, circuitry, interfaces and/or code, which may be operable to perform some degree of beam steering, which may be perpendicular to the scan plane direction. The ultrasound probe 104 may comprise a two dimensional (2D) or three dimensional (3D) array of piezoelectric elements. In an exemplary embodiment of the invention, the ultrasound probe 104 may comprise a three dimensional (3D) array of elements that is operable through suitable delays to steer a beam in the desired spatial 3D direction with a desired depth of focus. The ultrasound probe 104 may comprise a group of transmit transducer elements 106 and a group of receive transducer elements 108, that normally constitute the same elements. The ultrasound probe 104 may comprise a sensor 112 for coordinating with a needle emitter 14 to track the position of a surgical needle 10. The sensor 112 can correspond with a permanent magnet, an electromagnetic coil, an optical source, or any suitable emitter 14 that corresponds with the sensor 112 to form a tracking system.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals 107 into a region of interest (e.g., human, animal, underground cavity, physical structure and the like). The transmitted ultrasonic signals 107 may be back-scattered from structures in the object of interest, like blood cells or tissue, as well as any surgical instruments in the region or object of interest, like a surgical needle 10, to produce echoes 109. The echoes 109 are received by the receive transducer elements 108.

The group of receive transducer elements 108 in the ultrasound probe 104 may be operable to convert the received echoes 109 into analog signals, undergo sub-aperture beamforming by a receive sub-aperture beamformer 116 and are then communicated to a receiver 118.

The receiver 118 may comprise suitable logic, circuitry, interfaces and/or code that may be operable to receive and demodulate the signals from the receive sub-aperture beamformer 116. The demodulated analog signals may be communicated to one or more of the plurality of A/D converters 122.

The plurality of A/D converters 122 may comprise suitable logic, circuitry, interfaces and/or code that may be operable to convert the demodulated analog signals from the receiver 118 to corresponding digital signals. The plurality of A/D converters 122 are disposed between the receiver 118 and the receive beamformer 120. Notwithstanding, the invention is not limited in this regard. Accordingly, in some embodiments of the invention, the plurality of A/D converters 122 may be integrated within the receiver 118.

The receive beamformer 120 may comprise suitable logic, circuitry, interfaces and/or code that may be operable to perform digital beamforming processing on the signals received from the plurality of A/D converters 122. The resulting processed information may be converted back to corresponding RF signals. The corresponding output RF signals that are output from the receive beamformer 120 may be communicated to the RF processor 124. In accordance with some embodiments of the invention, the receiver 118, the plurality of A/D converters 122, and the beamformer 120 may be integrated into a single beamformer, which may be digital.

The RF processor 124 may comprise suitable logic, circuitry, interfaces and/or code that may be operable to demodulate the RF signals. In accordance with an embodiment of the invention, the RF processor 124 may comprise a complex demodulator (not shown) that is operable to demodulate the RF signals to form I/Q data pairs that are representative of the corresponding echo signals. The RF or I/Q signal data may then be communicated to an RF/IQ buffer 126.

The RF/IQ buffer 126 may comprise suitable logic, circuitry, interfaces and/or code that may be operable to provide temporary storage of the RF or I/Q signal data, which is generated by the RF processor 124.

The user input module 130 may be utilized to input patient data, surgical instrument data, scan parameters, settings, configuration parameters, change scan mode, and the like. In an exemplary embodiment of the invention, the user input module 130 may be operable to configure, manage and/or control operation of one or more components and/or modules in the ultrasound system 100. In this regard, the user input module 130 may be operable to configure, manage and/or control operation of transmitter 102, the ultrasound probe 104, the transmit beamformer 110, the receiver 118, the receive beamformer 120, the RF processor 124, the RF/IQ buffer 126, the user input module 130, the signal processor 132, the image buffer 136, and/or the display system 134.

The signal processor 132 may comprise suitable logic, circuitry, interfaces and/or code that may be operable to process ultrasound scan data (i.e., RF signal data or IQ data pairs) for generating an ultrasound image for presentation on a display system 134. The signal processor 132 is operable to perform one or more processing operations according to a plurality of selectable ultrasound modalities on the acquired ultrasound scan data. In an exemplary embodiment of the invention, the signal processor 132 may be operable to perform compounding, motion tracking, and/or speckle tracking. Acquired ultrasound scan data may be processed in real-time during a scanning session as the echo signals 109 are received. Additionally or alternatively, the ultrasound scan data may be stored temporarily in the RF/IQ buffer 126 during a scanning session and processed in less than real-time in a live or off-line operation. In the exemplary embodiment, the signal processor 132 may comprise a spatial compounding module 140.

The ultrasound system 100 may be operable to continuously acquire ultrasound scan data at a frame rate that is suitable for the imaging situation in question. Typical frame rates range from 20-70 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several seconds worth of frames of ultrasound scan data. The frames of ultrasound scan data are stored in a manner to facilitate retrieval thereof according to its order or time of acquisition. The image buffer 136 may be embodied as any known data storage medium.

The spatial compounding module 140 is optional and may comprise suitable logic, circuitry, interfaces and/or code that may be operable to combine a plurality of steering frames corresponding to a plurality of different angles to produce a compound image. In an embodiment, the compounding provided by module 140 may include frames steered or directed at an angle to produce a stronger reflection from the needle 10 based on needle position and orientation information provided by the tracking system.

The signal processor 132 may comprise suitable logic, circuitry, interfaces and/or code that may be operable to process acquired tracking information (i.e., magnetic field strength data or any suitable tracking information from sensor 112 or 14) for determining a tracked position and orientation of a surgical instrument 10, and process ultrasound scan data (i.e., RF signal data or IQ data pairs) for determining a scanned position and orientation of surgical instrument 10 detected within the ultrasound scan data. The signal processor 132 may comprise suitable logic, circuitry, interfaces and/or code that may be operable to compare the tracked position and orientation of a surgical instrument 10 with the scanned position and orientation of the surgical instrument 10 to determine a calibration error, which can be an ultrasound system calibration error or a tracking system calibration error, for example. The signal processor 132 is operable to perform one or more processing operations to determine and compare tracked and scanned position and orientation information of a surgical needle 10. In the exemplary embodiment, the signal processor 132 may comprise a processing module 150.

The processing module 150 may comprise suitable logic, circuitry, interfaces and/or code that may be operable to handle processing of tracking data and ultrasound scan data to provide automatic needle recalibration detection by comparing a recognized needle 10 position and orientation in ultrasound data 109 with a tracked needle 10 position and orientation provided by a tracking system 14, 112. In this regard, the processing module 150 may comprise suitable logic, circuitry, interfaces and/or code that may be operable to handle processing the acquired tracking information (i.e., magnetic field strength data or any suitable tracking information from sensor 112 or 14) for calculating a needle position and orientation and/or for determining an ultrasound beam steering angle. Further, the processing module 150 may comprise suitable logic, circuitry, interfaces and/or code that may be operable to handle processing the ultrasound scan data acquired at the determined ultrasound beam steering angle, for example, for determining a scanned position and orientation of a needle 10 detected within the ultrasound scan data. In a representative embodiment, the scanned position and orientation of a needle 10 can be detected within the ultrasound scan data by pattern recognition or any suitable detection method, for example.

The processing module 150 may comprise suitable logic, circuitry, interfaces and/or code that may be operable to perform one or more processing operations to compute and compare the tracked and scanned position and orientation information of a surgical needle 10 to determine a tracking system and/or ultrasound system calibration error. In various embodiments, the processing module 150 may comprise suitable logic, circuitry, interfaces and/or code that may be operable to automatically recalibrate (e.g., if the calibration error is below some threshold level), prompt a user with an option to automatically recalibrate, and/or prompt a user to recalibrate the tracking system 14, 112 by first removing the surgical needle 10 from the sensor range of tracking system 14,112 (e.g., if the determined calibration error exceeds a threshold).

In an exemplary embodiment of the invention, X, Y, and Z coordinate positions of a needle emitter 14 with respect to the probe sensor(s) 112 can be determined in real-time by the signal processor 132 using tracking data, such as magnetic field strength data sensed by the probe sensor(s) 112. The position and orientation information determined by the signal processor 132, together with the length of the needle portion 12 and position of the needle emitter 14 with respect to the distal insertion end as known by or input into the signal processor 132, enable the signal processor 132 to accurately determine the position and orientation of the entire length of the surgical needle 10 with respect to the probe sensor(s) 112 in real-time. Because the signal processor 132 is able to determine the position and orientation of the needle 10 with respect to the probe sensor(s) 112, the position and orientation of the needle 10 with respect to an ultrasound image can also be accurately determined by the signal processor 132. The probe sensor(s) 112 are configured to continuously detect tracking data from the emitter 14 of the needle 10 during operation of the ultrasound system 100. This enables the signal processor 132 to optionally determine an ultrasound beam steering angle with better likelihood for acquiring ultrasound scan data capturing the needle 10 (e.g., by increasing the beam angle relative to the expected needle position), and to continuously update the tracked position and orientation of the needle 10 for use in comparing the tracked position and orientation of the needle 10 with a scanned position and orientation of the needle 10 to determine a calibration error.

The ultrasound scan data acquired at the determined ultrasound beam steering angle, for example, can be provided to the processing module 150. In certain embodiments, the processing module 150 may apply pattern recognition algorithms, among other things, to the acquired ultrasound data to calculate a scanned position and orientation of the needle 10 detected within the ultrasound scan data. The processing module 150 can be configured to continuously track the position and orientation of the needle 10 in the acquired ultrasound data for comparison with the continuously detected tracking data, such that a calibration error is determined in substantially real-time. In a representative embodiment, if the determined calibration error is less than a pre-determined threshold (i.e., the error is relatively small), a recalibration procedure can be automatically initiated or a user prompt may be given for initiating an automatic procedure for recalibrating the tracking system. If the determined calibration error exceeds a pre-determined threshold, by contrast, a user prompt may be given to repeat the initial calibration procedure after removing the needle 10 from the surgical environment such that the permanent magnet 14 is out of range of the probe sensor(s) 112, for example.

In operation and in an exemplary embodiment of the invention, one or more sensors 112 of an ultrasound probe 104 configured to detect a magnetic field of the magnetic emitter 14 included with a needle 10 are calibrated with the emitter 14 out of range of the sensor(s) 112. After the tracking system 14, 112 is calibrated, the probe 104 is placed against the patient skin, transmits an ultrasound beam 107 to a target within a patient, and receives ultrasound echoes 109 used to generate an ultrasound image. The ultrasound image of the target can be depicted on the display 134 of the ultrasound system 100. A signal processor 132 of the ultrasound system 100 generates an ultrasound image that comprises a representation of the needle 10 based on the acquired ultrasound scan data. The representation may be an image of the needle 10 when the needle 10 is in-plane of the ultrasound image data, for example. Additionally and/or alternatively, the representation can be a virtual representation of the needle 10 overlaid on the ultrasound image of the target when, for example, the needle 10 is out-of-plane of the ultrasound image data or is simply not generating a strong reflection due to a shallow angle of the transmitted beams relative to the needle 10. In various embodiments, the ultrasound image can be generated by compounding the ultrasound image data of the target.

The system 100 is configured to detect the position and orientation of the surgical needle 10. Particularly, one or more sensors 112 of the probe 104 is configured to detect a magnetic field of the magnetic emitter 14 included with the needle 10. The sensor(s) 112 are configured to spatially detect the magnetic emitter 14 in three dimensional space. As such, during operation of the ultrasound system 100, magnetic field strength data emitted by the magnetic emitter 14 and sensed by the one or more sensors 112 is communicated to a processing module 150 of a signal processor 132 that continuously computes the real-time position and/or orientation of the needle 10. The real-time tracked position and/or orientation of the needle 10 may be used to determine a beam steering angle, for example. The determined beam steering angle is optionally applied by an ultrasound probe 104 to perform an ultrasound scan better capturing the needle 10. The acquired ultrasound scan data is processed by the processing module 150 of the signal processor 132 to determine a scanned position and/or orientation of the needle 10. The scanned position and/or orientation of the needle 10 are compared by the processing module 150 with the tracked position and/or orientation of the needle 10 to determine a calibration error of the tracking system 14, 112 or the ultrasound system 100. If the calibration error of the tracking system 14, 112 or ultrasound system 100 exceeds a pre-determined threshold, a recalibration procedure can be initiated. In various embodiments, the recalibration procedure can be an automatic procedure for recalibrating the tracking system based on the scanned position and/or orientation of the needle 10 or recalibrating the ultrasound system 100 based on the tracked position and/orientation of the needle 10. In certain embodiments, the ultrasound system 100 can notify a user of the determined calibration error and/or prompt the user with an option for proceeding with automatic recalibration based on the scanned or tracked position and/or orientation of the needle 10. In an embodiment, the recalibration procedure may be a procedure where the ultrasound system 100 can prompt a user to remove the needle 10 and re-perform the tracking system calibration prior to restarting the medical procedure.

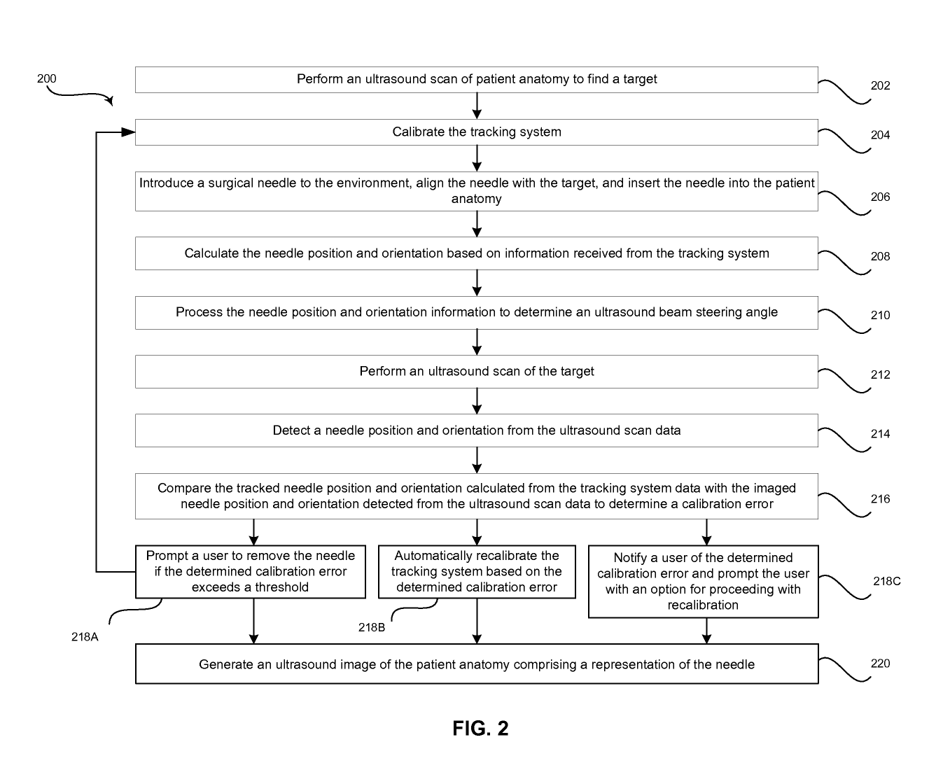

FIG. 2 is a flow chart illustrating exemplary steps that may be utilized for providing automatic needle recalibration detection by comparing a recognized needle 10 position and orientation in ultrasound data 109 with a tracked needle 10 position and orientation provided by a tracking system 14, 112, in accordance with an embodiment of the invention. Referring to FIG. 2, there is shown a flow chart 200 comprising exemplary steps 202 through 220. Certain embodiments of the present invention may omit one or more of the steps, and/or perform the steps in a different order than the order listed, and/or combine certain of the steps discussed below. For example, some steps may not be performed in certain embodiments of the present invention. As a further example, certain steps may be performed in a different temporal order, including simultaneously, than listed below.

In step 202, the ultrasound probe 104 in the ultrasound system 100 may be operable to perform an ultrasound scan of patient anatomy to find a target, such that the probe 104 is positioned at the target.

In step 204, a tracking system may be calibrated. For example, in a tracking system comprising a permanent magnet emitter 14 coupled to or within a surgical needle 10 and one or more sensors 112 coupled to or within a probe 104, the needle 10 may be removed from the surgical environment so that the tracking system can be calibrated to remove or zero-out ambient magnetic fields detected by the sensor(s) 112.

In step 206, a surgical needle 10 can be introduced to the surgical environment, aligned with a target, and inserted into the patient anatomy while the probe remains stationary.

In step 208, a processing module 150 of a signal processor 132 of the ultrasound system 100 can calculate a tracked position and orientation of the needle 10 based at least in part on information received from the tracking system 14, 112. For example, in a tracking system comprising a permanent magnet emitter 14 coupled to or within a surgical needle 10 and one or more sensors 112 coupled to or within a probe 104, the probe sensor(s) 112 can detect the magnet field change caused by the introduction of the permanent magnet emitter 14 of the needle 10 into the surgical environment. The probe sensor(s) 112 may provide the magnetic field strength data to the processing module 150 of the signal processor 132 such that X, Y, and Z coordinate positions of a needle emitter 14 with respect to the probe sensor(s) 112 can be determined in real-time. In particular, the position and orientation information determined by the processing module 150, together with the length of the needle portion 12 and position of the needle emitter 14 with respect to the distal insertion end as known by or input into the processing module 150, enable the processing module 150 to accurately determine the position and orientation of the entire length of the surgical needle 10 with respect to the probe sensor(s) 112 in real-time.

In step 210, the processing module 150 of the signal processor 132 can process the tracked needle position and orientation to optionally determine an ultrasound beam steering angle that has better odds of providing a strong needle 10 reflection than the steering angle used for otherwise imaging the region or object of interest.

In step 212, the ultrasound probe 104 in the ultrasound system 100 may be operable to perform an ultrasound scan of patient anatomy. In an embodiment, the ultrasound scan can optionally be based on the determined ultrasound beam steering angle. For example, the processing module 150 of the signal processor 132 can apply the ultrasound beam steering angle to the transmitter 102 and/or transmit beamformer 110 to acquire ultrasound scan data that includes the needle 10 by controlling the emission of the ultrasonic transmit signals 107 into a region of interest.

In step 214, a scanned position and orientation of the needle 10 can be detected from the ultrasound scan data acquired at step 212. For example, the processing module 150 of the signal processor 132 may apply pattern recognition processing, or any suitable detection processing, to determine the X, Y, and Z coordinate positions of a needle 10 with respect to the ultrasound scan data in substantially real-time. As another example, an operator can provide a user input via a user input module 130 and/or a touch screen display 134 to identify the scanned position and orientation of the needle 10 in displayed ultrasound data. In various embodiments, a user can trace an image of the needle 10 on the touch screen display 134 to identify the scanned position and orientation of the needle 10, for example.

In step 216, the processing module 150 of the signal processor 132 of the ultrasound system 100 may compare the scanned position and/or orientation of the needle 10 with the tracked position and/or orientation of the needle 10 to determine a calibration error of the tracking system 14, 112 or the ultrasound system 100. For example, an ultrasound system calibration error can be a scaling error in the ultrasound scan data that may be caused by speed-of-sound variation in different tissue types.

In steps 218A-C, the ultrasound system 100 is operable to provide a recalibration procedure based on the calibration error determined at step 216 and a pre-determined threshold. In various embodiments, the pre-determined threshold may be selectable by a user or based on a procedure, for example. In this regard, in one embodiment of the invention, in step 218A, the ultrasound system 100 is operable to provide a user prompt to repeat the initial calibration procedure in step 204 after removing the needle 10 from the surgical environment such that the permanent magnet 14 is out of range of the probe sensor(s) 112 if the calibration error exceeds a pre-determined threshold. In another embodiment of the invention, in step 218B, the ultrasound system 100 is operable to automatically recalibrate the tracking system or the ultrasound system based on the determined calibration error if the calibration error is less than a pre-determined threshold. In step 218C, the ultrasound system 100 is operable to notify a user of the determined calibration error and/or prompt the user with an option for proceeding with automatic recalibration based on the determined calibration error if the calibration error is less than a pre-determined threshold. In various embodiments, one or more of steps 218A-C can be alternative recalibration procedures. In certain embodiments, one or more recalibration procedures can be selected from the plurality of recalibration procedures 218A-C before, during, and/or after performing method 200, for example.

In step 220, the signal processor 132 can generate an ultrasound image of the patient anatomy comprising a representation of the needle 10. For example, the representation may include an image of the needle 10 when the needle 10 is in-plane of the ultrasound scan data. As another example, the representation can include a virtual representation of the needle 10 overlaid on the ultrasound image of the target when the needle is in-plane and/or out-of-plane of the ultrasound scan data. In various embodiments, spatial compounding module 140 can generate the ultrasound image by compounding the ultrasound scan data of the target. In certain embodiments, the compounded image may include frames steered or directed at an angle to produce a stronger reflection from the needle 10 based on needle position and orientation information provided by the tracking system.

Aspects of the present invention have the technical effect of providing automatic surgical instrument recalibration detection by comparing a recognized surgical instrument 10 position and orientation in ultrasound data 109 with a tracked surgical instrument 10 position and orientation provided by a tracking system 14, 112. In accordance with various embodiments of the invention, a method 200 comprises calibrating 204 a tracking system comprising a sensor 112 and an emitter 14, the sensor 112 and the emitter 14 being attached to or within a different one of a probe 104 of an ultrasound system 100 and a surgical instrument 10, respectively.

The method 200 comprises determining 208, by a processor 132, 150 of the ultrasound system 100, a tracked position and orientation of the surgical instrument 10 based at least in part on tracking information emitted by the emitter 14 of the tracking system and detected by the sensor 112 of the tracking system. The method 200 comprises performing 212, by the probe 104 of the ultrasound system 100, an ultrasound scan 107 to acquire ultrasound scan data 109. The method 200 comprises determining 214 a scanned position and orientation of the surgical instrument 10 based on the ultrasound scan data 109. The method 200 comprises comparing 216, by the processor 132, 150, the tracked position and orientation of the surgical instrument 10 with the scanned position and orientation of the surgical instrument 10 to determine a calibration error.

In various embodiments, the surgical instrument 10 is a needle. In certain embodiments, the method 200 comprises providing a user prompt 218A to repeat the calibrating the tracking system step 204 if the calibration error exceeds a threshold. In a representative embodiment, the method 200 comprises automatically recalibrating 218B the tracking system based on the scanned position and orientation of the surgical instrument 10 if the calibration error is less than a threshold. In various embodiments, the method 200 comprises providing a user option 218C for proceeding with automatic recalibration of the tracking system based on the scanned position and orientation of the surgical instrument 10 if the calibration error is less than a threshold. In certain embodiments, the user option 218C comprises tracing an image of the surgical instrument 10 on a touch screen display 134 to proceed with automatic recalibration of the tracking system.

In a representative embodiment, the scanned position and orientation of the surgical instrument 10 is determined by pattern recognition processing applied to the ultrasound scan data 109. In various embodiments, the emitter 14 is a permanent magnet coupled to the surgical instrument 12 and the tracking information comprises magnetic field strength. In certain embodiments, the tracking system is calibrated with the surgical instrument 10 outside a surgical environment, and comprising introducing the surgical instrument 10 into the surgical environment such that the sensor 112 of the calibrated tracking system detects the magnetic field strength emitted by the permanent magnet 14.

In certain embodiments, the method 200 comprises generating 220, by the processor 132, an ultrasound image based on the ultrasound scan data 109, the ultrasound image comprising a representation of the surgical instrument 10. In a representative embodiment, the representation of the surgical instrument 10 is an image of the surgical instrument 10 when the surgical instrument 10 is in-plane of the ultrasound scan data 109, and a virtual representation of the surgical instrument 10 overlaid on the ultrasound image when the surgical instrument 10 is out-of-plane of the ultrasound scan data 109.

In another embodiment, a virtual representation of the surgical instrument 10 overlaid on the ultrasound system is continuously displayed even when the surgical instrument is in-plane of the ultrasound scan data and clearly visible in the displayed image. By displaying the virtual needle 10 representation even when the reflected needle 10 representation is clearly visible, an operator is better able to identify a small calibration error that might not have been detected by the processor 132, 150. If that were to happen, the operator could use the user input module 130 to prompt a recalibration of the tracking system. In some embodiments, the user could even trace the reflected image of the needle 10 on a touch screen display 134 to help the system better determine the position and orientation of the needle 10 for more accurate recalibration of the tracking system without having to remove the needle 10 from the region or object of interest.

Various embodiments provide a system comprising an ultrasound device 100 that comprises a processor 132, 140, 150 and a probe 104. The processor 132, 150 is operable to determine a position and orientation of a surgical instrument 10 based on tracking information emitted by an emitter 14 of a tracking system and detected by a sensor 112 of the tracking system. The sensor 112 and the emitter 14 are attached to or within a probe 104 of the ultrasound device 100 and the surgical instrument 10, respectively. The processor 132, 150 is operable to determine a scanned position and orientation of the surgical instrument 10 based on ultrasound scan data 109 acquired by the probe 104. The processor 132, 150 is operable to compare the tracked position and orientation of the surgical instrument 10 with the scanned position and orientation of the surgical instrument 10 to determine a calibration error. The processor 132, 150 is operable to adjust the tracked position and orientation of the surgical instrument 10 or the scanned position and orientation of the surgical instrument 10 based on the calibration error.

In certain embodiments, the surgical instrument 10 is a needle. In a representative embodiment, the emitter 14 is a permanent magnet coupled to the needle 10. In various embodiments, the tracking information comprises magnetic field strength. In certain embodiments, a user prompt to calibrate the tracking system is provided if the calibration error exceeds a threshold. In a representative embodiment, the tracking system is automatically calibrated based on the scanned position and orientation of the surgical instrument 10 if the calibration error is less than a threshold. In various embodiments, a user option for proceeding with automatic calibration of the tracking system based on the scanned position and orientation of the surgical instrument 10 is provided if the calibration error is less than a threshold.

Certain embodiments provide a non-transitory computer readable medium having stored a computer program comprising at least one code section that is executable by a machine for causing the machine to perform steps 200 disclosed herein. Exemplary steps 200 may comprise calibrating 204 a tracking system comprising a sensor 112 and an emitter 14. The sensor 112 and the emitter 14 may be attached to or within a probe 104 of an ultrasound system 100 and a surgical instrument 10, respectively. The steps 200 can comprise determining 208 a tracked position and orientation of the surgical instrument 10 based at least in part on tracking information emitted by the emitter 14 of the tracking system and detected by the sensor 112 of the tracking system. The steps 200 may comprise performing 212 an ultrasound scan 107 to acquire ultrasound scan data 109. The steps 200 can comprise determining 214 a scanned position and orientation of the surgical instrument 10 based on the ultrasound scan data 109. The steps 200 may comprise comparing 216 the tracked position and orientation of the surgical instrument 10 with the scanned position and orientation of the surgical instrument 10 to determine a calibration error.

In a representative embodiment, the steps 200 can comprise providing a user prompt 218A to repeat the calibrating the tracking system step if the calibration error exceeds a threshold. In various embodiments, the steps 200 may comprise automatically recalibrating 218B the tracking system based on the scanned position and orientation of the surgical instrument 10 if the calibration error is less than a threshold. In certain embodiments, the steps 200 can comprise providing a user option 218C for proceeding with automatic recalibration of the tracking system based on the scanned position and orientation of the surgical instrument 10 if the calibration error is less than a threshold.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments of the invention may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for providing automatic needle recalibration detection by comparing a recognized needle position and orientation in ultrasound data with a tracked needle position and orientation provided by a tracking system.

Accordingly, the present invention may be realized in hardware, software, or a combination of hardware and software. The present invention may be realized in a centralized fashion in at least one computer system, or in a distributed fashion where different elements are spread across several interconnected computer systems. Any kind of computer system or other apparatus adapted for carrying out the methods described herein is suited. A typical combination of hardware and software may be a general-purpose computer system with a computer program that, when being loaded and executed, controls the computer system such that it carries out the methods described herein.

The present invention may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A method, comprising:
calibrating a tracking system comprising a sensor and an emitter, the sensor and the emitter being attached to or within a probe of an ultrasound system and a surgical instrument, respectively;
determining, by a processor of the ultrasound system, a tracked position and orientation of the surgical instrument based at least in part on tracking information emitted by the emitter of the tracking system and detected by the sensor of the tracking system;
performing, by the probe of the ultrasound system, an ultrasound scan to acquire ultrasound scan data;
determining a scanned position and orientation of the surgical instrument based on the ultrasound scan data; and
comparing, by the processor, the tracked position and orientation of the surgical instrument with the scanned position and orientation of the surgical instrument to determine a calibration error.

2. The method according to claim 1, wherein the surgical instrument is a needle.

3. The method according to claim 1, comprising providing a user prompt to repeat the calibrating the tracking system step if the calibration error exceeds a threshold.

4. The method according to claim 1, comprising automatically recalibrating the tracking system based on the scanned position and orientation of the surgical instrument if the calibration error is less than a threshold.

5. The method according to claim 1, comprising providing a user option for proceeding with automatic recalibration of the tracking system based on the scanned position and orientation of the surgical instrument if the calibration error is less than a threshold.

6. The method according to claim 5, wherein the user option comprises tracing an image of the surgical instrument on a touch screen display to proceed with automatic recalibration of the tracking system.

7. The method according to claim 1, wherein the scanned position and orientation of the surgical instrument is determined based on a user input.

8. The method according to claim 7, wherein the user input comprises tracing an image of the surgical instrument on a touch screen display.

9. The method according to claim 1, wherein the scanned position and orientation of the surgical instrument is determined by pattern recognition processing applied to the ultrasound scan data.

10. The method according to claim 9, wherein the emitter is a permanent magnet coupled to the surgical instrument and the tracking information comprises magnetic field strength.

11. The method according to claim 10, wherein the tracking system is calibrated with the surgical instrument outside a surgical environment, and comprising introducing the surgical instrument into the surgical environment such that the sensor of the calibrated tracking system detects the magnetic field strength emitted by the permanent magnet.

12. The method according to claim 1, comprising generating, by the processor, an ultrasound image based on the ultrasound scan data, the ultrasound image comprising a representation of the surgical instrument.

13. The method according to claim 12, wherein the representation of the surgical instrument comprises at least one of:
an image of the surgical instrument when the surgical instrument is in-plane of the ultrasound scan data, and
a virtual representation of the surgical instrument overlaid on the ultrasound image when the surgical instrument is in-plane or out-of-plane of the ultrasound scan data.

14. The method according to claim 1, wherein the ultrasound scan is performed based on the tracked position and orientation of the surgical instrument.

15. The method according to claim 1, comprising at least one of:
automatically recalibrating the ultrasound system based on the tracked position and orientation of the surgical instrument if the calibration error is less than a threshold, and
providing a user option for proceeding with automatic recalibration of the ultrasound system based on the tracked position and orientation of the surgical instrument if the calibration error is less than a threshold.

16. A system, comprising:
an ultrasound device comprising:
a processor operable to:
determine a position and orientation of a surgical instrument based on tracking information emitted by an emitter of a tracking system and detected by a sensor of the tracking system, the sensor and the emitter being attached to or within a probe of the ultrasound device and the surgical instrument, respectively,
determine a scanned position and orientation of the surgical instrument based on ultrasound scan data acquired by a probe, and
compare the tracked position and orientation of the surgical instrument with the scanned position and orientation of the surgical instrument to determine a calibration error; and
adjust at least one of the tracked position and orientation of the surgical instrument and the scanned position and orientation of the surgical instrument based on the calibration error.

17. The system according to claim 16, wherein:
the surgical instrument is a needle,
the emitter is a permanent magnet coupled to the needle, and
the tracking information comprises magnetic field strength.

18. The system according to claim 16, wherein a user prompt to calibrate the tracking system is provided if the calibration error exceeds a threshold.

19. The system according to claim 16, wherein the tracking system is automatically calibrated based on the scanned position and orientation of the surgical instrument if the calibration error is less than a threshold.

20. The system according to claim 16, wherein a user option for proceeding with automatic calibration of the tracking system based on the scanned position and orientation of the surgical instrument is provided if the calibration error is less than a threshold.

21. The system according to claim 16, wherein at least one of:
the ultrasound system is automatically calibrated based on the tracked position and orientation of the surgical instrument if the calibration error is less than a threshold, and
a user option for proceeding with automatic calibration of the ultrasound system based on the tracked position and orientation of the surgical instrument is provided if the calibration error is less than a threshold.

22. A non-transitory computer readable medium having stored thereon, a computer program having at least one code section, the at least one code section being executable by a machine for causing the machine to perform steps comprising:
calibrating a tracking system comprising a sensor and an emitter, the sensor and the emitter being attached to or within a probe of an ultrasound system and a surgical instrument, respectively;
determining a tracked position and orientation of the surgical instrument based at least in part on tracking information emitted by the emitter of the tracking system and detected by the sensor of the tracking system;
performing an ultrasound scan to acquire ultrasound scan data;
determining a scanned position and orientation of the surgical instrument based on the ultrasound scan data; and
comparing the tracked position and orientation of the surgical instrument with the scanned position and orientation of the surgical instrument to determine a calibration error.

23. The non-transitory computer readable medium according to claim 22, comprising providing a user prompt to repeat the calibrating the tracking system step if the calibration error exceeds a threshold.

24. The non-transitory computer readable medium according to claim 22, comprising automatically recalibrating the tracking system based on the scanned position and orientation of the surgical instrument if the calibration error is less than a threshold.

25. The non-transitory computer readable medium according to claim 22, comprising providing a user option for proceeding with automatic recalibration of the tracking system based on the scanned position and orientation of the surgical instrument if the calibration error is less than a threshold.

26. The non-transitory computer readable medium according to claim 22, comprising at least one of:
automatically recalibrating an ultrasound system based on the tracked position and orientation of the surgical instrument if the calibration error is less than a threshold, and
providing a user option for proceeding with automatic recalibration of an ultrasound system based on the tracked position and orientation of the surgical instrument if the calibration error is less than a threshold.

27. The non-transitory computer readable medium according to claim 22, wherein the ultrasound scan is performed based on the tracked position and orientation of the surgical instrument.
